(19) United States
(12) Patent Application Publication
(10) Pub. No.: US 2005/0014665 A1
(43) Pub. Date: Jan. 20, 2005

(54) Solid detergent composition

(76) Inventors: Suresh Murigeppa Nadakatti, Banglore (IN); Vijay Mukund Naik, Banglore (IN)

Correspondence Address: UNILEVER;PATENT DEPARTMENT, 45 RIVER ROAD, EDGEWATER, NJ, 07020, US

(21) Appl. No.: 10/489,974

(22) PCT Filed: Aug. 23, 2002
(86) PCT No.: PCT/EP02/09415

(30) Foreign Application Priority Data
Sep. 18, 2001 (IN) 895/MUM/2001

(51) Int. Cl. A61K 7/50

(52) U.S. Cl. 510/141

(57) ABSTRACT

A continuous process for casting comprising the steps of i. filling a continuous tube of flexible material with a melt of the castable composition, where the tube acts as a sleeve to the composition, such that a desired cross section area of the filled sleeve is obtained that is independent of its perimeter ii. solidifying and simultaneously shaping the said melt by cooling the said filled sleeve in or on a suitable mould.

### TECHNICAL FIELD

The present invention relates to a process for continuous casting of a solid detergent composition.

### BACKGROUND AND PRIOR ART

Soap or non-soap detergent articles are traditionally produced either by extrusion or by casting routes.

The processing/finishing of a detergent article via the extrusion route often involves various operations such as homogenisation, shear working, and forming into a suitable shape. A plodder or extruder is usually employed to take care of part of the shaping operation. The product from the extruder may be cut into logs and/or billets which are subsequently stamped and shaped into tablets or bars. The extrusion of soap or detergent mass is a continuous operation.

The casting route for detergent bars is traditionally employed for example for producing highly transparent soaps. The most widely employed technology involves many manufacturing operations such as casting of molten soap into logs of desired cross section in moulds, cooling, log ejection, log maturation, billeting, chamfering, billet maturation and stamping to produce individual transparent soap tablets or bars.

Casting allows for high formulation flexibility, for instance high levels of liquid benefit agents and water can be incorporated into the composition. Highly transparent bars can be processed by casting.

However, conventional casting is a batch/semi-continuous operation and therefore is highly labour/capital intensive as compared to extrusion which is a continuous operation. Continuous casting is employed for rapid setting materials such as steel and glass under continuous “drawing” conditions. However such a process can not be employed for non quick setting materials such as soaps and detergents. Thus it would be useful to make the casting of non-quick setting materials a continuous operation and less labour/capital intensive.

EP 321,179 (Unilever), discloses a method of casting soap or detergent wherein liquid or semi-liquid soap is filled into a pack made of flexible film, such that the material occupies the whole of the pack. The pack is then tightly sealed to exclude air and the material is allowed to set in the pack to obtain cast in pack tablets. This process will help in producing a continuous string of packed soap sachets. The ability to manipulate shape of the tablet in such a process is limited. Minor shape manipulations are achieved by pressurising the pack that is essentially made of heat or pressure extendable film and in general the shape obtained is in the form of a cushion. Such internal pressure can be developed by externally compressing the sealed sachet. The process, although referred to as a continuous method of casting, requires sealing and pressurising of individual sachets, and thus would significantly slow down the throughput rates. In the absence of the heat extendable or shrinkable polymers there would be formation of wrinkles on the cast tablets.

It has now been possible to have a simple, economical, continuous process for casting of non-quick setting materials, at high through put rates. It is further possible to obtain a desired shape of tablet, free of wrinkles and air cavities, without having to use heat extendable polymers, and without pressurising the filled liquid. In the process of the invention, the melt of the castable composition is filled into a tube made of flexible material where the tube acts as a sleeve to the composition, such that desired cross section area of the filled sleeve is obtained that is independent of the perimeter of the sleeve. The filled sleeve is then shaped during cooling and solidification. The process produces cast-in-sleeve logs that can be cut into billets/tablets and optionally flow wrapped.

The tube made of the flexible material does not necessarily have a circular cross section. The sleeve provides a means for pulling, conveying and shaping of the liquid. The melt of the composition is filled into the tube, acting as a sleeve for the composition, with a control on cross section area of the filled sleeve which is independent of the perimeter of the sleeve. This fact in turn, can be exploited to obtain the desired shape during cooling and solidification. One of the means for achieving desired cross section area of the filled sleeve independent of the perimeter is to provide a guide that constrains the sleeve during filling. The other means could be to fill to less than 100% of the internal volume of a substantially air free tube. Internal pressure is not required for achieving desired shapes of tablets. The increased throughput is achieved by avoiding the sealing of the individual sachets and producing continuously shaped cast-in-sleeve logs thereby simulating the extrusion process. The shaping is achieved by placing the sleeve filled with the liquid composition on a suitable rigid mould during cooling and solidification. The sleeve filled with the liquid composition is pulled either horizontally or vertically and cooled to bring about solidification.

The above described continuous casting process is suitable for manufacturing any non quick setting materials such as soaps, detergent tablets, deodorants, confectioneries, etc.

### OBJECTS OF THE INVENTION

Thus, it is an object of the invention to provide a simple, economical, continuous process for in-sleeve-casting of non quick-setting materials whereby the shape of the cast product is controlled.

### DESCRIPTION OF THE INVENTION

According to one aspect of the present invention there is provided a continuous process for casting comprising the steps of:

i. filling a continuous tube of flexible material, optionally formed online with a melt of the castable composition, where the tube acts as a sleeve to the composition, such that the desired cross section area of the filled sleeve is obtained that is independent of its perimeter; and

ii. solidifying and simultaneously shaping the said melt by cooling the said filled sleeve in or on a suitable mould.

It is particularly preferred that the desired cross section area of the filled sleeve independent of the perimeter of the filled sleeve is achieved by providing a guide that constrains the sleeve during filling or by filling to less than 100% of the internal volume of a substantially air free tube.

According to a preferred aspect of the present invention there is provided a continuous process for casting comprising the steps of:

i. filling a continuous tube of a flexible material, optionally formed online, with a melt of the castable composition, where the tube acts as a sleeve to the composition, and simultaneously conveying through a cross section constraining guide to achieve desired cross-section area of the filled sleeve that is independent of its perimeter

ii. solidifying and simultaneously shaping the said melt by cooling the said filled sleeve in/on a suitable mould

iii. cutting the said continuously formed shaped and solidified cast composition into logs/billets/tablets.

iv. optionally flow wrapping the said logs/billets/tablets

According to a more preferred aspect of the present invention there is provided a continuous process for casting comprising the steps of:

(i) filling to less than 100% of the internal volume of a substantially air free continuous tube of a flexible material, optionally formed online, the said tube sealed at the bottom end, with a melt of the castable composition and sealing the filling end to obtain a cast-in-sleeve melt

(ii) solidifying and simultaneously shaping by cooling the said melt to obtain cast-in-sleeve log of the composition

(iii) optionally cutting the said shaped and solidified cast-in-sleeve log into billets/tablets

(iv) optionally flow wrapping the said logs/billets/tablets.

According to a most preferred aspect of the present invention there is provided a continuous process for casting comprising the steps of:

(i) filling a continuous tube of flexible material formed online and sealed at the bottom end, with a melt of the castable composition, where the tube acts as a sleeve to the composition, and simultaneously conveying through a cross section constraining guide to achieve desired area of cross section of the filled sleeve that is independent of the perimeter

(ii) sealing the filling end of the filled tubular sleeve without air entrapment to obtain a cast-in-sleeve melt

(iii) solidifying and simultaneously shaping the said melt by cooling the said filled sleeve on a suitable mould to obtain a cast-in-sleeve log

(iv) cutting the said shaped and solidified cast composition into billets/tablets.

(v) optionally flow wrapping the said logs/billets/tablets

The different products that can be cast using the above process are for example soaps, detergents, deodorants or confectioneries. However, the process is particularly preferred for home and personal care compositions such as soap and detergent bars or tablets.

The cast-in-sleeve detergent tablet can be transparent or non-transparent and formed from any suitable formulation known in the art. The composition can comprise only soap as the active or can be in combination with non-soap detergent actives. The billets or tablets of the detergent composition can optionally be dehydrated.

The particularly preferred detergent composition comprises

i. 10-60% saturated fatty acid soap

ii. 0-40% non-soap detergent active

iii. 20-80% water

iv. optionally salting in electrolytes, solubilizers such as polyols, benefit agents, etc.

### DETAILED DESCRIPTION OF THE INVENTION

The essential feature of the invention is that the through put of the casting process can be significantly enhanced and desired shapes of tablets can be obtained by filling a melt of the composition into a tube of flexible material that acts as a sleeve to the composition, optionally formed online, such that desired cross section area of the filled sleeve is achieved independent of the perimeter of the sleeve. The filled sleeve is shaped during cooling and solidification to obtain cast-in-sleeve log. The increased throughput is achieved by avoiding sealing of the individual sachets and producing continuously shaped cast-in-sleeve logs thereby simulating the extrusion process. It is possible to use any flexible material without having to use special polymers such as heat extendable or shrinkable or heat sealable polymers to obtain tablets with desired shapes. The shaping is achieved by placing the tubular sleeve filled with the liquid in or on a suitable rigid mould during cooling and solidification.

The nature of the invention, its objects and advantages will be more apparent from the following detailed description and these are non-limiting details of the different aspects of the invention.

The tube:

The tube used in the casting process can be either preformed or formed online from a suitable flexible material. The tube acts as a sleeve to the composition. The tube is preferably formed online. In a preferred method of forming the tube online, a suitable flexible material is enveloped around a rigid forming unit and sealed vertically to form the tube. The tube need not necessarily be of circular cross section. The tube provides a means for pulling, conveying and shaping of the composition.

It is preferred that a tube substantially free of air is obtained prior to filling the melt of the castable composition, if guide is not used to achieve desired cross section of the filled sleeve independent of perimeter of the sleeve. One of the ways of obtaining a tube substantially free of air is to minimise or avoid air leakage into the tube during its formation from the flexible material. In one of the preferred means of achieving desired cross section area of the filled sleeve independent of the perimeter of the sleeve, the melt of the castable composition is filled to less than 100% of the internal volume of a tube substantially free of air and then sealed to obtain a cast-in-sleeve melt. The tubular sleeve containing melt of the composition is then shaped during cooling and solidification.

The tube is made from a flexible material such as polymer, rubber, paper, fabric, etc. The flexible material should be chosen such that the melt of the composition being filled into the tube should not leak under hydrostatic pressure. Polymers are especially suitable to make the tube. It is not essential that the polymer be heat sealable, heat shrinkable or heat extendable. It is also possible to use laminated films. Polymers suitable for making the tube include poly(vinyl chloride), nylon, polyester, polystyrene, cellulosic polymers and polyethylene. Less expensive materials like polyethylene and paper coated with polyethylene are especially preferred.

Guide:

One of the other means of achieving desired cross section area of filled sleeve independent of the perimeter of the sleeve is to provide a guide that constrains the tube during filling. Thus, the volume occupied by the composition per unit length of the tube is lower when the guiding mechanism is in place. In the process the guide must be provided until the composition is cooled to obtain the desired three dimensional shape which otherwise will change under hydrostatic pressure.

As an alternative it is possible to seal the sleeve by sealing through the liquid while the sleeve is still guided to obtain a log of cast-in-sleeve melt. The guide also ensures that the air bubbles stay out of the log of the cast-in-sleeve melt at the time of top sealing. In this case it is not essential to maintain the guide until shaping but it is essential to have the composition in the melt form to give it the desired shape. If the composition solidifies before shaping it is possible to remelt and then shape the same.

Without being limited by the same, the guiding means is provided by constraining the filled tubular sleeve between two movable rigid surfaces prior to top sealing. Examples of such surfaces include flat plates, rods, curved plates etc.

The Composition:

The different products that can be cast by using the above process may be a soap, detergent, deodorant or confectioneries. However, the process is particularly preferred for home and personal care compositions such as soap and detergent bars.

The Detergent Composition:

Any castable detergent composition disclosed in prior art is suitable for the process of the invention. The particularly preferred composition comprises:

i. 10-60% saturated fatty acid soap

ii. 0-40% detergent active

iii. 20-60% water

iv. optionally salting-in electrolytes, polyols, benefits 10 agents etc.

Saturated Fatty Acid Soap:

The saturated fatty acid soap is preferably selected from one or more salts of C₆-C₂₄ fatty acids. The soap employed may for example be a sodium, potassium, magnesium, aluminium, calcium or lithium salt of saturated fatty acids. It is especially preferred to have soap obtained as sodium or potassium salt of saturated fatty acid.

The saturated fatty acid soap in the composition is preferably 10-60% by weight of the composition, most preferred from 15 to 40% by weight.

Detergent Active:

The compositions according to the invention optionally comprise detergent actives. The detergent active can be non-soap detergent actives or the salts of unsaturated fatty acids. Non soap detergent actives are suitably selected from anionic, non-ionic, cationic, amphoteric or zwitterionic surfactants or their mixtures. The detergent active is suitably used in an amount of from 0 to 40% by weight of the composition, most preferred 5 to 35% by weight of the composition.

Examples of suitable detergent active species are given in the following well-known textbooks: (i) “Surface Active Agents”, Volume I by Schwartz and Perry, (ii) “Surface Active Agents and Detergents”, Volume II by Schwartz, Perry and Berch, (iii) “Handbook of Surfactants”, M. R. Porter, Chapman and Hall, New York, 1991.

Salting-In Electrolytes:

Salting-in electrolytes for use in the composition are selected from those listed in the ‘Hofmeister’ or ‘Lyotropic’ series. The salting-in electrolytes are generally those wherein the lyotropic number for the anion of the electrolyte is >10. Some examples of anions with lyotropic number >10 are NO₂⁻, ClO₃⁻, Br⁻, NO₃⁻, ClO₄⁻, I⁻, CNS⁻, C₆H₅SO₃—, C₆H₄CH₃SO₃⁻ and Cr₂O₇²⁻. The preferred examples of salting-in electrolytes for use in compositions according to the present invention are alkali metal salts of the above mentioned anions. The most preferred examples of the salting-in electrolytes for use in compositions according to the present invention are sodium toluene sulphonate, sodium cumene sulphonate and sodium xylene sulphonate. Further examples of salting-in electrolytes may be selected form those described in (i) Collins, K. D.; Washabaugh, M. W. Quart. Rev. Biophys., 1985, 18, 323; (ii) Schuster. P, Zundel. G and Sandorfy. C, 1976, ‘The Hydrogen Bond’, Recent developments in theory and experiments, Vol. III, North-Holland Publishing Co. Amsterdam, New York, Oxford.

Liquid Benefit Agents:

According to a preferred aspect of the invention, liquid skin benefit materials such as moisturisers, emollients, sunscreens, anti ageing compounds are incorporated in the composition. Examples of moisturisers and humectants include polyols, glycerol, cetyl alcohol, carbopol 934, ethoxylated castor oil, paraffin oils, lanolin and its derivatives. Silicone compounds such as silicone surfactants like DC3225C (Dow Corning) and/or silicone emollients, silicone oil (DC-200 Ex-Dow Corning) may also be included. Sun-screens such as 4-tertiary butyl-4′-ethoxy dibenzoylmethane (available under the trade name PARSOL 1789 from Givaudan) and/or 2-ethyl hexyl methoxy cinnamate (available under the trade name PARSOL MCX from Givaudan) or other UV-A and UV-B sun-screens.

Solubilisers:

Solubilisers suitable for use in the detergent composition include monohydric and polyhydric alcohols such as propylene glycol, sorbitol, glycerine etc.

Optional Ingredients:

Other optional ingredients such as hair conditioning agents, fillers, colour, perfume, opacifier, preservatives, one or more water insoluble particulate materials such as talc, kaolin, polysaccharides and other conventional ingredients may be incorporated in the composition.

Cooling and Shaping of the Composition:

The melt may have any suitable temperature, for example up to 120° C., most preferred from 40° C. to 90° C. The melt of the composition in the tubular sleeve (cast-in-sleeve composition) is passed through a cooling tunnel to bring about solidification. The product is simultaneously shaped in the cooling tunnel. The shaping is achieved by providing a conveyor belt in the cooling tunnel, said belt being flat or curved, thus imparting the required shape to the solidified composition. For example, a flat conveyor belt can be used to obtain a flat bottom, convex top shaped cast-in-sleeve product.

It is also possible to pressurise the tubular sleeve filled with the melt of the composition between two plates to obtain a desired shape.

Another means of achieving the desired shape is to place the tubular sleeve filled with the melt of the composition in or on a suitable mould which in turn is placed on the conveyor belt of the cooling tunnel. Lettering or decorative motifs can be provided during shaping in the cooling tunnel.

The solidified and shaped composition so obtained is then cut to obtain cast-in-sleeve logs or billets or tablets. Logs so obtained can be further cut into billets or tablets. Optionally the logs or billets or tablets can be flow wrapped.

The nature of the invention, its objects and advantages will be more apparent from the ensuing description made with relation to non-limiting exemplary examples of the above identified various aspects of the invention.

### EXAMPLES

### Example 1

The Detergent Composition

A mixture containing fatty acid soap, non-soap detergent, salting-in electrolyte, and water as described in Table 1 was mixed in a two litre capacity round bottom flask. The batch temperature was raised to 80° C. The batch temperature was maintained at 80° C. so that a clear pourable liquid was obtained.

TABLE 1

| | Wt % of the |
|---|---|
| Component | Composition |
| Sodium stearate | 20 |
| Sodium lauryl ether sulphate | 15 |
| Sodium toluene sulphonate | 10 |
| Water | 55 |
| Total | 100 |

### Example 2

Continuous Casting Process to obtain a Cast-In-Sleeve Product without Sealing the Sleeve

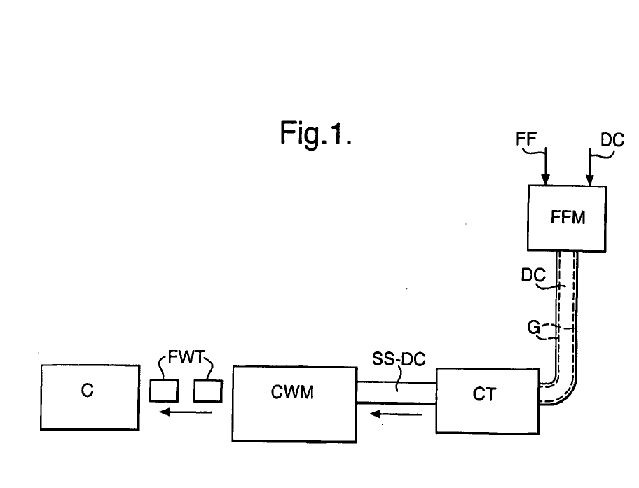

FIG. 1 shows a schematic representation of the continuous casting process. The flexible film (FF) is continuously formed in the forming and filling machine (FFM) to obtain the tube. Simultaneously the melt of the detergent composition (DC) is filled into the said tube while guiding the tube by a guiding means (G). The melt of the detergent composition in the sleeve is then solidified and simultaneously shaped by passing through the cooling tunnel (CT) provided with a flat conveyor belt. The continuously formed, solidified and shaped detergent composition (SS-DC) is then cut into billets and flow wrapped using the cut and wrap machine (CWM). The flow wrapped tablets (FWT) are then cartoned. The detergent tablet had a flat bottom and a convex top that has advantages of: (i) convenient to hold in hand and (ii) easy to apply on body or onto fabric.

### Example 3

Demonstration of the Need for the Guide for Obtaining Desired Shapes

i. Casting without the Guide:

A pre-formed tube made from 80 micron thick polyethylene with 19 Cm perimeter and 80 Cm length was filled with the melt of the detergent composition as described in Example 1 up to a height of 70 Cm and sealed through the liquid at a length of 65 Cm. Two such cast-in-sleeve logs were made and one of the logs was placed on a flat surface (Example 3a) and the other on a mould with a concave surface with radius of curvature 6 Cm (Example 3b), cooled to bring about solidification. The log was cut into 10 Cm length tablets.

ii. Casting with a Guide:

A pre-formed tubular sleeve made from 80 micron thick polyethylene with 19 Cm perimeter and 80 Cm length was placed in a guide made up of two parallel flat acrylic sheets of width 8 Cm, length of 60 Cm and with a spacing of 4.5 Cm between them. The sleeve was filled with the melt of the detergent composition as described in Example 1 up to a height on 70 Cm and sealed through the liquid at a length of 65 Cm. Two such cast-in-sleeve logs were made and one of the logs was placed on a flat surface (Example 3c) and the other on a mould with a concave surface with radius of curvature 6 Cm (Example 3d), cooled to bring about solidification. The log was cut into 10 Cm length tablets.

The details of the shape of the tablets obtained are presented in Table 2.

TABLE 2

| Examples | Shape of the tablet |
|---|---|
| Example 3a | Cylinder with a circular cross section |
| Example 3b | Cylinder with a circular cross section |
| Example 3c | Substantially flat bottomed with a convex dome |
| Example 3d | Cylinder with an elliptical cross section. |

The data presented show that when the guide is used during casting the shape of the tablet can be controlled and the tablets obtained would be free of wrinkles.

1. A continuous process for casting soaps, detergents, deodorants or confectionaries, comprising the steps of:
i. filling a continuous tube of flexible material with a melt of the castable composition, where the tube acts as a sleeve to the composition, such that a desired cross section area of the filled sleeve is obtained that is independent of its perimeter
ii. solidifying and simultaneously shaping the said melt by cooling the said filled sleeve in or on a suitable mould, and
wherein the desired cross section area is achieved by a guide that constrains the sleeve during filling to less than 100% of the internal volume of a substantially air-free tube.

2. A continuous process for casting comprising the steps of:
i. filling a continuous tube of a flexible material, optionally formed online, with a melt of the castable composition,.where the tube acts as a sleeve to the composition, and simultaneously conveying through a cross section constraining guide to achieve desired cross-section area of the filled sleeve that is independent of its perimeter
ii. solidifying and simultaneously shaping the said melt by cooling the said filled sleeve in or on a suitable mould
iii. cutting the said continuously formed shaped and solidified cast composition into logs or billets or tablets.

3. A continuous process for casting comprising the steps of:
i. filling a continuous tube of flexible material formed online and sealed at the bottom end, with a melt of the castable composition, where the tube acts as a sleeve to the composition, and simultaneously conveying through a cross section constraining guide to achieve desired area of cross section of the filled sleeve that is independent of the perimeter
ii. sealing the filling end of the filled tubular sleeve without air entrapment to obtain a cast-in-sleeve melt
iii. solidifying and simultaneously shaping the said melt by cooling the said filled sleeve on a suitable mould to obtain a cast-in-sleeve log
iv. cutting the said shaped and solidified cast composition into billets or tablets.

4. Process according to claim 1 wherein the detergent has a composition comprising:
i. 10-60% saturated fatty acid soap
ii. 0-40% non-soap detergent active
iii. 20-80% water
